(19) United States
(12) Patent Application Publication
(10) Pub. No.: US 2026/0051323 A1
(43) Pub. Date: Feb. 19, 2026

(54) INTERACTIVE CARE ROBOT

(71) Applicant: ROBOCARE CO., LTD., Seongnam-si (KR)

(72) Inventors: Deok Won LEE, Seongnam-si (KR); Kook Sung JUN, Seongnam-si Gyeonggi-do (KR); Won Jun LEE, Seongnam-si (KR)

(21) Appl. No.: 19/369,442

(22) Filed: Oct. 27, 2025

Related U.S. Application Data
(63) Continuation of application No. PCT/KR2024/010803, filed on Jul. 25, 2024.

(30) Foreign Application Priority Data
May 10, 2024 (KR) 10-2024-0061614

(51) Int. Cl. G10L 15/22; G06N 3/008; G06V 10/12; G06V 40/16; G10L 15/183; G10L 25/63; G10L 25/66; G16H 80/00

(52) U.S. Cl. CPC G10L 15/22; G06N 3/008; G10L 15/183; G16H 80/00; G06V 10/12; G06V 40/161; G06V 40/172; G06V 40/174; G10L 25/63; G10L 25/66

(57) ABSTRACT

An interactive care robot according to an embodiment of the present disclosure includes: at least one sensor including a microphone configured to receive voice conversations with a user; a memory configured to store instructions; and a processor operatively connected to the memory and configured to execute the instructions. The processor is configured to: generate conversation history information based on the conversations with the user; generate user information and classify the user information into predetermined categories; set a priority of a care service to be provided to the user based on the classified user information and the conversation history information; and select a care service to be provided to the user based on the set priority and control components of the interactive care robot to perform the selected care service.

### TECHNICAL FIELD

The present disclosure relates to a care robot that is configured to provide a care service based on a conversation between the care robot and a user.

### BACKGROUND

In recent years, robotic technology has advanced significantly. In particular, innovative developments have been made in the field of care robots configured to support individuals in their daily lives. Such care robots are configured to facilitate users' daily activities by assisting with household chores, supporting health management, or the like. Earlier developed care robots were mainly configured to perform simple and repetitive tasks or to operate according to commands previously set by users. For example, they were limited to moving along pre-programmed paths or providing basic interactions in response to specific commands.

However, such conventional robots were limited to operating based on programmed scenarios or algorithms and thus had considerable difficulty in timely recognizing and responding to individual user demands or changes in circumstances. In practice, it has become necessary for robots to operate in a more flexible and intelligent manner in order to recognize dynamic factors, such as changes in a user's health condition, emotional fluctuations, or changes in preferences, and to respond appropriately thereto. This requires robots not only to execute commands, but also to detect nonverbal signals or emotional states of users and to provide services based on such detected information. However, earlier developed robots were not equipped with the advanced technology needed to automatically analyze complex user states and thus were limited in their ability to respond appropriately to specific user requests. As a result, the level of personalization in the user experience was low, which limited satisfaction with the services provided by the robots.

In order to solve the foregoing problems, the need for interactive care robots has emerged. There is a demand for care robots that can continuously collect information through conversations with users, identify their needs based on the collected information, and provide personalized service. This allows the robots to more accurately understand the users' daily patterns, preferences, and psychological states, and to improve the quality of the users' lives by providing customized services based on such understanding.

### DISCLOSURE OF THE INVENTION

### Problems to be Solved by the Invention

The present disclosure is conceived to provide a care robot that can more specifically identify a user's needs from conversation content between the care robot and the user and provide appropriate care services.

The present disclosure is conceived to provide a care robot that can analyze conversation history between the care robot and a user and set care services to be preferentially provided to the user based on the analysis, thereby improving the user's satisfaction.

The present disclosure is conceived to provide a care robot that can set a storage period for user information derived from conversation history between the care robot and a user, thereby enabling the care robot to perform conversations with the user in a more natural manner.

The present disclosure is conceived to provide a care robot that can analyze a user's emotions from conversations between the care robot and the user, thereby enabling the care robot to provide more appropriate care services.

The present disclosure is conceived to provide a care robot that can analyze a user's health condition from conversations between the care robot and the user, thereby enabling the care robot to provide care services according to the analyzed health condition.

The present disclosure is conceived to provide a care robot that can analyze a user's cognitive ability from conversations between the care robot and the user and store the analyzed information for later use.

However, the problems to be solved by the present disclosure are not limited to the above-described problems. There may be other problems to be solved by the present disclosure.

### Means for Solving the Problems

As a means for achieving the above-described technical problems, an aspect of the present disclosure provides an interactive care robot, including: at least one sensor including a microphone configured to receive voice conversations with a user; a memory configured to store instructions; and a processor operatively connected to the memory and configured to execute the instructions. The processor is configured to: generate conversation history information based on the conversations with the user received through the microphone; generate user information from the conversation history information and classify the user information into predetermined categories; set a priority of a care service to be provided to the user based on the classified user information and the conversation history information; and select a care service to be provided to the user based on the set priority and control components of the interactive care robot to perform the selected care service.

The above-described aspects are provided by way of illustration only and should not be construed as liming the present disclosure. Besides the above-described embodiments, there may be additional embodiments described in the accompanying drawings and the detailed description.

### Effects of the Invention

According to any one of the above-described means for solving the problems of the present disclosure, the present disclosure can more specifically identify a user's needs from conversation content between a care robot and the user and provide appropriate care services.

The present disclosure can analyze conversation history between the care robot and the user and set care services to be preferentially provided to the user based on the analysis, thereby improving the user's satisfaction.

The present disclosure can set a storage period for user information derived from conversation history between the care robot and the user, thereby enabling the care robot to perform conversations with the user in a more natural manner.

The present disclosure can analyze the user's emotions from conversations between the care robot and the user, thereby enabling the care robot to provide more appropriate care services.

The present disclosure can analyze the user's health condition from conversations between the care robot and the user, thereby enabling the care robot to provide care services according to the analyzed health condition.

The present disclosure can analyze the user's cognitive ability from conversations between the care robot and the user and use the analyzed information.

### BRIEF DESCRIPTION OF THE DRAWINGS

FIG. 1 is a configuration view of an interactive care robot and a care system according to an embodiment of the present disclosure.

FIG. 2 is a configuration view of the interactive care robot according to an embodiment of the present disclosure.

FIG. 3 to FIG. 5 are diagrams illustrating a process of generating sensing information by the interactive care robot according to an embodiment of the present disclosure.

FIG. 6 and FIG. 7 are diagrams illustrating a process of controlling operations of the interactive care robot according to an embodiment of the present disclosure.

FIG. 8 is a diagram illustrating a process of generating situation information by the interactive care robot according to an embodiment of the present disclosure.

FIG. 9 and FIG. 10 are diagrams illustrating a process of analyzing a user's emotion by an emotion information generator according to an embodiment of the present disclosure.

FIG. 11 is a diagram illustrating a process of analyzing the user's cognitive ability by a cognitive information generator according to an embodiment of the present disclosure.

### BEST MODE FOR CARRYING OUT THE INVENTION

Hereafter, example embodiments will be described in detail with reference to the accompanying drawings so that the present disclosure may be readily implemented by those skilled in the art. However, it is to be noted that the present disclosure is not limited to the example embodiments but can be embodied in various other ways. In the drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected” another element and an element being “electronically connected” to another element via another element. Further, it is to be understood that the terms “comprises,” “includes,” “comprising,” and/or “including” means that one or more other components, steps, operations, and/or elements are not excluded from the described and recited systems, devices, apparatuses, and methods unless context dictates otherwise; and is not intended to preclude the possibility that one or more other components, steps, operations, parts, or combinations thereof may exist or may be added.

Throughout this document, the term “unit” may refer to a unit implemented by hardware, software, and/or a combination thereof. As examples only, one unit may be implemented by two or more pieces of hardware or two or more units may be implemented by one piece of hardware.

Throughout this document, a part of an operation or function described as being carried out by a terminal or device may be implemented or executed by a device connected to the terminal or device. Likewise, a part of an operation or function described as being implemented or executed by a device may be so implemented or executed by a terminal or device connected to the device.

The functionality of the elements disclosed herein may be implemented using circuitry or processing circuitry which includes general purpose processors, special purpose processors, integrated circuits, ASICs (“Application Specific Integrated Circuits”), conventional circuitry and/or combinations thereof which are configured or programmed to perform the disclosed functionality.

Processors are considered processing circuitry or circuitry as they include transistors and other circuitry therein. In the disclosure, the circuitry, units, or means are hardware that carry out or are programmed to perform the recited functionality.

The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

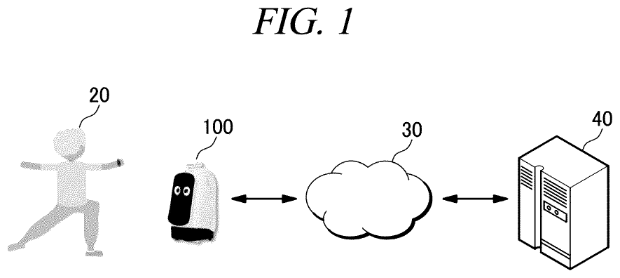

FIG. 1 is a diagram illustrating an interactive care robot 100 and a care system according to an embodiment of the present disclosure.

Referring to FIG. 1, a care system may include the interactive care robot 100 and a server 40 that are configured to provide a care service to a user 20.

The interactive care robot 100 illustrated in FIG. 1 may be connected to the server 40 via a network 30. As shown in FIG. 1, the interactive care robot 100 and the server 40 may be connected to the network 30 simultaneously or with a time interval. The network 30 refers to a connection structure that enables information exchange between nodes, such as devices and servers, and includes LAN (Local Area Network), WAN (Wide Area Network), Internet (WWW: World Wide Web), a wired or wireless data communication network, a telecommunication network, a wired or wireless television network, and the like. Examples of the wireless data communication network may include 3G, 4G, 5G, 3GPP (3rd Generation Partnership Project), LTE (Long Term Evolution), WIMAX (World Interoperability for Microwave Access), Wi-Fi, Bluetooth communication, infrared communication, ultrasonic communication, VLC (Visible Light Communication), LiFi, and the like, but may not be limited thereto.

The interactive care robot 100 may be a robot equipped with its own means of mobility, and may include a camera for imaging the user 20, a microphone for receiving sounds input from the user, a display for presenting information to the user, and a speaker for outputting sounds to the user. In addition to the camera and the microphone, the interactive care robot 100 may further include various sensors configured to detect surrounding conditions, such as temperature, humidity, illuminance, and pressure. The interactive care robot 100 may also include an integrated controller configured to control the above-described devices. The integrated controller may include a memory and a processor, thereby functioning as a device having computational processing capability.

In an embodiment of the present disclosure, the camera of the interactive care robot 100 may image an object to generate image type information. For example, an RGB (Red Green Blue) camera configured to generate pixel images having RGB properties may be used. The camera may also be a depth camera that provides distance information of an object or an infrared camera capable of capturing images in dark environments. The camera may perform still photography to generate a single image of the user 20, or may perform video recording to generate a video composed of a plurality of frames.

The memory may be a device configured to store information, and may include various types of memories, such as a high-speed random access memory, a magnetic disk storage device, a flash memory device, and other non-volatile solid-state memory devices. In the interactive care robot 100, the memory may be implemented in the form of a database.

The interactive care robot 100 may generate a prompt to be input into a language model based on information collected from the user 20, determine a care service to be provided to the user based on an output from the language model, and control components of the interactive care robot 100 to provide the determined care service to the user. A more detailed description thereof will be provided below.

For example, the components may include a speaker for voice guidance or music playback, a display for presenting information, a robot mover for robot locomotion, or a robotic arm (manipulator) for gripping an object, but are not limited thereto.

The user 20 may be a person who receives care services from the interactive care robot 100, and may typically refer to a patient or an elderly person who has difficulty in moving. However, the user 20 is not limited thereto and may include various persons who need to use the interactive care robot 100.

The server 40 may include a memory in which a plurality of modules is stored, a processor connected to the memory and configured to respond to the plurality of modules and to process service information provided to the interactive care robot 100 or action information for controlling the service information, a communication means, and a user interface (UI) display means.

In the present disclosure, the server 40 may correspond to an external server capable of operating a language model. The information processed by the server 40 may include robot-collected information, IoT-collected information, cloud-collected information, and available service information of the interactive care robot 100. Such information may be converted into service information or action information needed to instruct and coordinate the behavior of the interactive care robot 100.

The interactive care robot 100 may be linked to an external server including a cloud-based language model via the network 30. The external server may perform complex natural language processing tasks, and may generate appropriate linguistic responses based on data received from the interactive care robot 100. The interactive care robot 100 may utilize the generated responses to perform interactions with the user more naturally and effectively. The language model of the server 40 may provide the interactive care robot 100, in text form, with information on the user 20's situation and types of care services to be provided by the interactive care robot 100 to the user, in response to data input as prompts from the interactive care robot 100. To this end, the server 40 may analyze and process data generated from the plurality of modules. In this process, the communication means of the server 40 may enable continuous data exchange with the interactive care robot 100, and the UI display means may be designed to allow the user or administrator to monitor the server's status and perform necessary operations.

Such a structure ensures a smooth flow of information between the server 40 and the external server and enables the provision of customized care services to the user through complex data processing operations and response generation processes.

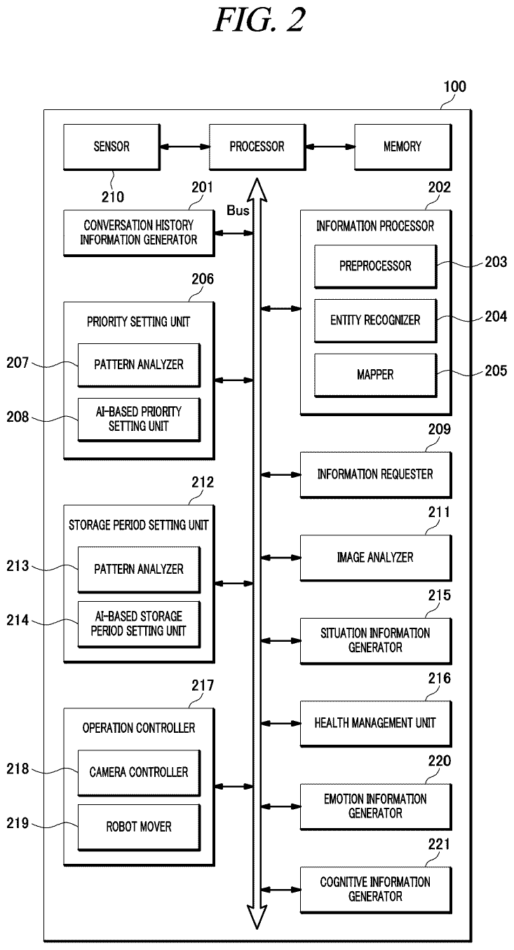

FIG. 2 is a configuration view of the interactive care robot 100 according to an embodiment of the present disclosure.

Referring to FIG. 2, the interactive care robot 100 may include a conversation history information generator 201, an information processor 202, a priority setting unit 206, an information requester 209, a sensor 210, an image analyzer 211, a storage period setting unit 212, a situation information generator 215, a health management unit 216, an operation controller 217, an emotion information generator 220, and a cognitive information generator 221.

The information processor 202 may include a preprocessor 203, an entity recognizer 204, and a mapper 205.

The priority setting unit 206 may include a pattern analyzer 207 and an AI-based priority setting unit 208, and the storage period setting unit 212 may include a pattern analyzer 213 and an AI-based storage period setting unit 214.

The operation controller 217 may include a camera controller 218 and a robot mover 219.

The conversation history information generator 201, the information processor 202, the priority setting unit 206, the information requester 209, the sensor 210, the image analyzer 211, the storage period setting unit 212, the situation information generator 215, the health management unit 216, the operation controller 217, the emotion information generator 220, the cognitive information generator 221, the information processor 202, the preprocessor 203, the entity recognizer 204, the mapper 205, the pattern analyzer 207, the AI-based priority setting unit 208, the pattern analyzer 213, the AI-based storage period setting unit 214, the camera controller 218, and the robot mover 219 shown in FIG. 2 may be logical components separated to describe the functional features of the present disclosure. The functional operations of these components (“units”) described throughout the whole document may be specifically implemented by the processor of the integrated controller. This is achieved by executing one or more computer-executable instructions stored in the memory, including interactions with specific hardware components.

In an embodiment of the present disclosure, the care robot may include at least one sensor 210, a memory configured to store instructions, and a processor operatively connected to the memory and configured to execute the instructions. The processor may acquire robot-collected information related to the user by using the sensor 210, generate a prompt based on the robot-collected information, transmit the generated prompt to a language model, select a care service to be provided to the user based on an output received from the language model, and control the components of the care robot to perform the selected care service.

More specifically, the interactive care robot 100 may include the conversation history information generator 201 configured to generate conversation history information based on conversations with the user, the information processor 202 configured to generate user information from the conversation history information and classify the user information into predetermined categories, and the priority setting unit 206 configured to set a priority of a care service of the care robot to be provided to the user based on the classified user information and the conversation history information.

The information processor 202 may include the preprocessor 203 configured to extract utterance sentences of the user from the conversation history information and preprocess the extracted sentences, the entity recognizer 204 configured to derive entities from the preprocessed utterance sentences through an entity recognition algorithm and assign attributes to the entities, and the mapper 205 configured to map the entities within the utterance sentences based on the attributes of the entities and generate the user information.

The priority setting unit 206 may include the pattern analyzer 207 and the AI-based priority setting unit 208. The pattern analyzer 207 is configured to derive daily pattern information and preference information of the user from the conversation history information. The AI-based priority setting unit 208 includes a priority setting model that receives the user information, the daily pattern information, and the preference information to output a priority for the user information.

The interactive care robot 100 may further include the information requester 209 configured to request additional information from the user based on the classified user information. The information requester 209 may select at least part of the user information based on the priority, and may request the additional information based on the selected user information.

The interactive care robot 100 may further include the sensor 210 and the image analyzer 211. The sensor 210 is configured to generate sensing information based on at least one sensing device including the camera. The image analyzer 211 is configured to detect an object in an image captured by the camera and detect a place of capture, and to determine, based on features of the detected object and the place of capture, whether the detected object is a newly added object. When the detected object is determined to be a newly added object, the information requester 209 may request additional information about the detected object from the user.

The interactive care robot 100 may further include the storage period setting unit 212 configured to set, in the user information, a storage period indicating a duration to be reflected in the conversations with the user. The storage period setting unit 212 may include the pattern analyzer 213 configured to derive daily pattern information and preference information of the user from the conversation history information and the AI-based storage period setting unit 214 including a storage period setting model that receives the user information, the daily pattern information, and the preference information to output a storage period for the user information.

The interactive care robot 100 may further include the situation information generator 215 and the health management unit 216. The situation information generator 215 is configured to generate situation information about the surrounding conditions of the user based on the sensing information generated by the sensor 210. The health management unit 216 is configured to generate prescription information based on additional information input through the information requester 209 and the user information.

The interactive care robot 100 may further include the operation controller 217 configured to control at least one of the operation of the camera of the sensor and movements of the interactive care robot to allow the situation information generator 215 to generate situation information. The operation controller 217 may include the camera controller 218 and the robot mover 219. The camera controller 218 is configured to control at least one of vertical rotation, horizontal rotation, and height adjustment of the camera. The robot mover 219 is configured to move the interactive care robot 100 to change a capture location of the interactive care robot 100.

The interactive care robot 100 may further include the emotion information generator 220 configured to derive emotion information of the user from the conversation history information and the user's utterance information generated by the microphone.

The interactive care robot 100 may further include the cognitive information generator 221 configured to evaluate the user's cognitive ability from answer information input by the user in response to a cognitive test provided to the user.

In addition to the above-described components, the interactive care robot 100 may further include various components configured to check the user's state and provide appropriate care services.

For example, the interactive care robot 100 may include a display configured to present services provided to the user 20 and a speaker configured to output sounds and voice messages generated while the services are provided to the user 20.

The display serves as a type of liquid crystal display, and may output one or more of text, an image, or a video containing predetermined information. Herein, the predetermined information may include status information of the interactive care robot 100, such as communication signal strength information, remaining battery information, or wireless Internet ON/OFF information. The display may present content related to services to be described below, and may display, in text form, voice output through the speaker. For example, when the interactive care robot 100 provides voice corresponding to service-related content to the user while issuing an operation instruction, text such as “Raise your right arm higher” may be displayed on the display. Further, as will be described later, during a process in which the interactive care robot 100 performs user identification of the user 20, the display may output text, such as “Please come closer” or “Please face me directly”, to instruct the user to perform actions required for the identification.

The display may output text by repeating a single type of information described above, by alternating between a plurality of types, or by outputting specific information by default. For example, status information of the interactive care robot 100, such as communication signal strength information, remaining battery information, or wireless Internet ON/OFF information, may be continuously output as small text at the top or bottom of the display while other types of information may be alternately output.

As described above, the display may output one or more of images or videos. In this case, to enhance visibility, it is preferable for the display to be implemented as a large, high-resolution liquid crystal display, rather than one that only outputs text. As will be described later, the display may be configured on an outer surface or inside the care robot, or may be provided as a separate device external to the interactive care robot 100.

Meanwhile, the display may be positioned on the front side of the interactive care robot 100. Thus, the user 20 facing the front of the interactive care robot 100 can view the content presented on the display together with the interactive care robot 100.

The speaker may output various sounds including voice. Herein, the voice is auditory information output by the interactive care robot 100 for interaction with the user. The type of voice can be set by using a media-specific application installed on the user 20's device (not shown) or by directly controlling the interactive care robot 100.

For example, the type of voice output through the speaker may be selected from various voices, such as a male voice, a female voice, an adult's voice, and a child's voice, and the language may also be selected, such as Korean, English, Japanese, and French.

The speaker not only outputs voice but also functions as a typical speaker for general sound output. For example, if the user 20 wants to listen to music through the interactive care robot 100, the music may be output through the speaker. If a video is displayed on the display, sound synchronized with the video may be output through the speaker.

Hereinafter, the operations of the conversation history information generator 201, the information processor 202, and the priority setting unit 206 of the interactive care robot 100 will be described in more detail.

The conversation history information generator 201 may generate conversation history information about conversations between the user and the interactive care robot 100. Herein, the conversation history information may be text information in which voice conversations between the user and the interactive care robot 100 are converted into text. The conversations between the user and the interactive care robot 100 may be carried out based on an STT (Speech To Text) algorithm, in which the interactive care robot 100 receives sounds generated by the user's utterances through the microphone, analyzes the content of the received sounds, and converts the analyzed content into text. Further, utterances of the interactive care robot 100 may be generated based on a TTS (Text To Speech) algorithm that converts the generated text into sound. That is, the user and the interactive care robot 100 may converse with each other by voice, and the content of the utterances in the conversation may be converted into text and generated as conversation history information. Furthermore, the conversations between the user and the interactive care robot 100 may also be carried out by text output on the display installed on the interactive care robot 100 or on an externally connected display, and text input by the user through touch or a keyboard.

The information processor 202 may generate user information from the conversation history information and classify the user information into predetermined categories. To this end, the information processor 202 may include: the preprocessor 203 configured to extract and preprocess utterance sentences of the user from the conversation history information; the entity recognizer 204 configured to derive entities from the preprocessed utterance sentences through an entity recognition algorithm and assign attributes to the entities; and the mapper 205 configured to map the entities within the utterance sentences based on the attributes of the entities and to generate the user information.

The information processor 202 may use artificial intelligence (AI) to derive entities from the user's utterance sentences. The entity recognizer 204 may apply an NER (Named Entity Recognition) algorithm to the preprocessed utterance sentences to identify specific words. The NER algorithm may refer to an algorithm for recognizing named entities. For example, in the user's utterance “My son's name is Chulsoo Kim,” the entity recognizer 204 may derive “Chulsoo Kim” as an entity through the NER algorithm and assign an attribute “name” to the entity. Further, the entity recognizer 204 may derive “son” as an entity from the utterance sentence and assign an attribute “family member” to the entity. As another example, in the utterance “These days I often go out to play golf,” the entity recognizer 204 may derive “these days” as an entity through the NER algorithm and assign an attribute “time” to the entity. Further, the entity recognizer 204 may derive “golf” as an entity and assign an attribute “exercise” to the entity. That is, the information processor 202 may derive important words as entities from utterance sentences and assign meanings or concepts corresponding to the derived words as attributes of the respective entities.

The mapper 205 may map the entities to each other based on the attributes of the recognized entities. For example, the mapper 205 may map and store “Chulsoo Kim” and “son” in a structure such as [family>son>name>Chulsoo Kim] based on the “name” attribute of “Chulsoo Kim” and the “family member” attribute of “son” that were derived from the example above. That is, the mapper 205 may map entities having similar attributes to each other.

The priority setting unit 206 may set a priority of a care service of the care robot 100 to be provided to the user based on the classified user information and the conversation history information.

The user information used to provide care services may vary depending on the user. In this case, there is a need for a process of providing care services based on user information having a higher priority among a plurality of types of user information. The user information having a higher priority may be information preferentially considered when the care robot 100 generates answers or provides care services during conversations with the user. Also, the user information having a higher priority may also be information for which the care robot 100 more frequently and specifically requests additional details from the user. For example, if the care robot 100 determines that the user likes cooking, it may ask more diverse and detailed questions about cooking and store more user information on the subject than on other subjects. The priority of user information may be determined based on rules or based on AI.

More specifically, the priority setting unit 206 may use a rule-based algorithm to set a priority of user information based on predetermined priorities for respective categories. For example, user information classified as personal information or family information may be given the highest priority based on rules. As another example, if the user is a student, user information related to their study or entrance exams may be given a high priority. As yet another example, if the user is in poor health, user information related to health may be given a high priority.

Alternatively, the priority setting unit 206 may set a priority of user information based on based on AI.

To this end, the priority setting unit 206 may include the pattern analyzer 207 and the AI-based priority setting unit 208. The pattern analyzer 207 is configured to derive daily pattern information and preference information of the user from the conversation history information. The AI-based priority setting unit 208 includes a priority setting model that receives the user information, the daily pattern information, and the preference information to output a priority for the user information.

That is, the priority may be automatically determined based on AI. When the user's daily patterns, preferences, and tastes are digitized, the priority setting unit 206 may set a priority of the user information through the priority setting model. The priority setting model may be implemented using machine learning or deep learning, and may also use a generative AI such as a Large Language Model (LLM).

The priority setting unit 206 may set priorities by using the priorities output from the AI-based priority setting unit 208 while also setting predetermined priorities for user information classified into predetermined categories. That is, priorities may be set using both AI and rules. For example, the priority setting unit 206 may give the highest priority to user information related to personal information or family information based on rules while allowing AI to set priorities for other user information by considering the user's tastes and preferences.

The information requester 209 may request additional information from the user based on the classified user information. More specifically, the information requester 209 may select at least part of the user information based on the priority, and may request the additional information based on the selected user information.

The information requester 209 may request additional information from the user either directly or indirectly. A direct request may refer to the care robot 100 directly asking the user for additional information related to a specific category via a text or voice conversation, such as “How many family members do you have?”, “What is your son's name?”, “What did you have for lunch today?”, “What is your current hobby?”, or “What movie did you watch recently?”

An indirect request may refer to the care robot 100 automatically extracting information that requires additional details from the user's utterance sentences. For example, if the user says, “Recently my legs hurt, so I have been visiting a Korean medicine clinic,” the care robot 100 may remember that the user visits a Korean medicine clinic and that the pain is in the legs. To perform an indirect request for information, the care robot 100 may a process of analyzing the user's utterance sentences using AI to identify specific words and semantically connect them. This process may be performed by sending related queries to a generative AI such as an LLM and using the output responses.

Meanwhile, the information requester 209 may also receive additional information based on the user's command. For example, if the user says, “Remember that there will be a birthday party at the senior center tomorrow morning,” the care robot 100 may store this sentence or these words as user information even if there is no predetermined related category or user information.

The information requester 209 may request additional information from the user for user information having a high priority. For example, if the care robot 100 recognizes that the name of the user's son is Chulsoo Kim and the related user information has a high priority, the care robot 100 may request additional information, such as the son's age or place of residence, from the user via a conversation, and may update the user information based on the user's responses.

In addition to conversations with the user, the information requester 209 may generate user information from information (e.g., new objects, people, or animals) collected by the camera of the care robot 100. The care robot 100 may store the information collected by the camera and later reconfirm it with the user via a conversation. For example, if the care robot 100 detects a new dog while moving around the home, it may store this information and later ask the user, “I saw a new dog. What is its name?” to update the user information related to the newly detected object. The care robot 100 may also recognize and utilize appliances, furniture, and other objects.

To this end, the care robot 100 may further include the sensor 210 and the image analyzer 211. The sensor 210 is configured to generate sensing information based on at least one sensing device including the camera. The image analyzer 211 is configured to detect an object in an image captured by the camera and detect a place of capture, and to determine, based on features of the detected object and the place of capture, whether the detected object is a newly added object.

When the detected object is determined to be a newly added object, the information requester 209 may request additional information about the detected object from the user.

The camera in the sensor 210 serves as the primary means of collecting information related to the user by capturing the user's face, walking scene, body shape, clothing, and the like. The images captured by the camera may play a key role in analyzing the user's identity. The sensor 210 may further include a location recognition device configured to generate information about the current capture location in addition to the images of the object. Meanwhile, as will be described later, when a smart map is generated based on the images captured by the camera, the care robot 100 may also generate information about the current capture location and capture angle according to its location on the smart map.

The image analyzer 211 may use an image processing model capable of recognizing an object in an image when image information is input. The image processing model may use an object recognition algorithm to recognize an object in the image. The image analyzer 211 may compare a current image with a past image captured from a similar location and direction to check for new objects and identify the types of new objects.

The storage period setting unit 212 may set, in the user information, a storage period indicating a duration to be reflected in a conversation with the user. The storage period setting unit 212 may set the storage period based on rules or based on AI.

That is, the storage period setting unit 212 may use a rule-based algorithm to set a storage period of user information based on predetermined storage periods for respective categories.

Criteria for setting the storage period for each type of user information by the storage period setting unit 212 may be predefined. For example, user information, such as the user's date of birth and gender, may not change, and thus may be set as permanent memory targets with permanent storage periods. Meanwhile, schedule information, preference information, and health information of the user may change over time, and thus may be set as temporary memory targets with temporary storage periods and assigned predetermined durations.

Alternatively, the storage period setting unit 212 may set storage periods by considering the user's situation, daily patterns, preferences, and tastes based on AI.

To this end, the storage period setting unit 212 may include the pattern analyzer 213 configured to derive daily pattern information and preference information of the user from the conversation history information and the AI-based storage period setting unit 214 including a storage period setting model that receives the user information, the daily pattern information, and the preference information to output a storage period for the user information.

The pattern analyzer 213 may generate daily pattern information and preference information based on the conversation history information or the user's situation information. The user's situation information may refer to information generated by the situation information generator 215. The pattern analyzer 213 may include generative artificial intelligence, such as a large language model (LLM), and may derive daily pattern information and preference information about the user's general lifestyle patterns, information, preferences, and tastes by inputting the conversation history information or the situation information into the language model.

The AI-based storage period setting unit 214 may include a storage period setting model configured to classify each type of user information as a permanent or temporary memory target when the user information, the daily pattern information, and the preference information are input, and to output an appropriate storage period when the information is classified as a temporary memory target.

User information set as a permanent memory target may be stored in the care robot 100 without change unless requested by the user. A temporary memory target may be checked by the care robot 100 after the set storage period elapses to determine whether the information is still valid, and may be updated accordingly. For example, if the care robot 100 stores “cold” as the user's health status, it may, after one week (the storage period), ask, “Have you recovered from your cold?” and may update the related user information based on the user's answer.

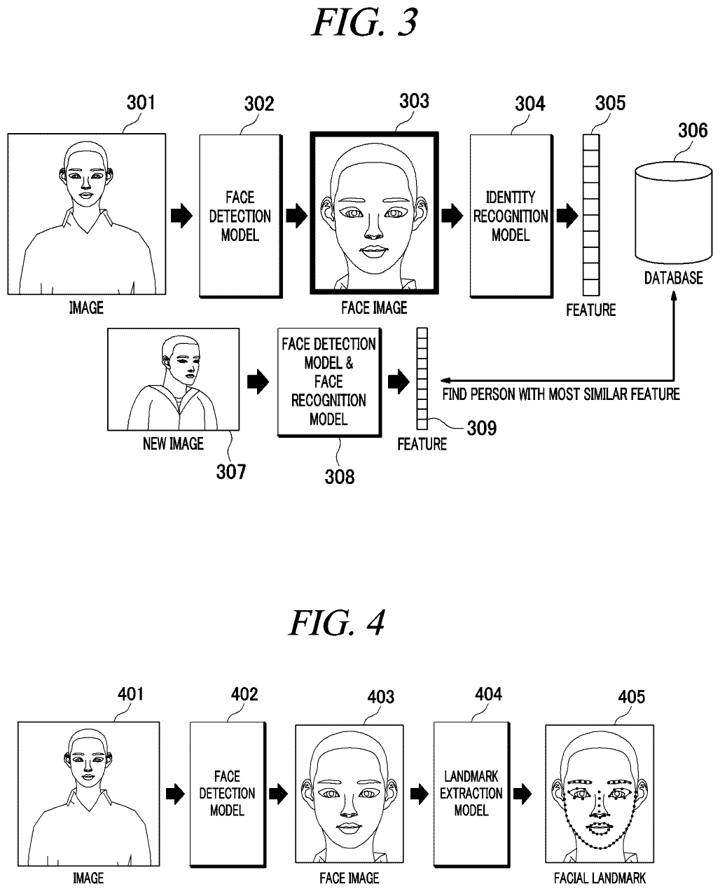

FIG. 3 to FIG. 5 are diagrams illustrating a process of generating sensing information by the interactive care robot according to an embodiment of the present disclosure.

The interactive care robot 100 may capture at least a part of the user 20's body (e.g., the face) through a camera included in the sensor 210.

The interactive care robot 100 may detect a face within an image being captured by the camera through a face detection model. The face detection model may be used in a similar way to the above-described object detection mode. Unlike the object detection model, which detects a specific object in an image, the face detection model may identify the location of a face in an image and generate a corresponding bounding box. As the face detection model, a convolutional neural network (CNN)-based detection model may be applied, and object detection algorithms, such as RCNN, Fast RCNN, YOLO, Single Shot Detector (SSD), Retina-Net, and Pyramid Net, may be used.

In order to improve the accuracy of results, information about the user's height, which is either previously input into the face detection model or estimated through a pose estimation model, may be provided to the care robot. Accordingly, the interactive care robot 100 may vertically rotate the camera toward the user's face to accurately recognize the face of the standing user even at a close distance. A more detailed description thereof will be provided below.

FIG. 3 is a diagram illustrating an identity recognition model 304 configured to apply the face detection model 302 to an image 301 and recognize the user's identity from a face image 303 generated based on the location of the face determined by the face detection model 302.

The identity recognition model 304 may extract features 305 from the face image 303 in the bounding box and store them in a database 306. Then, the identity recognition model 304 may perform identification based on the user's face image by comparing the features previously stored in the database with features 309 extracted from a new image 307 by applying the face detection model and the identity recognition model. As the identity recognition model 304, a CNN-based model may be applied, and algorithms, such as VGG-Face, FaceNet, OpenFace, DeepFace, and ArcFace, may be used.

Referring to FIG. 3, the face detection model 302 applies the face detection model 302 to the image of a person to generate a bounding box around a person's face in the image 301 and extract the face image 303, which is then input into the identity recognition model 304 to extract its features 305. The extracted features 305 are stored in the database 306. Thereafter, when the new image 307 is input, the features 309 are extracted by applying the above-described face detection model and identity recognition model 308 and then compared and matched with the features previously stored in the database 306 to recognize the identity.

To improve the accuracy of the results, the identity recognition model 304 may provide voice guidance to induce the user to face the camera of the interactive care robot 100. Accordingly, the interactive care robot 100 may generate a front image of the user 20's face to recognize the user's identity more accurately. A face orientation recognition model, which will be described later, may confirm whether the user's face is facing the camera based on its result data.

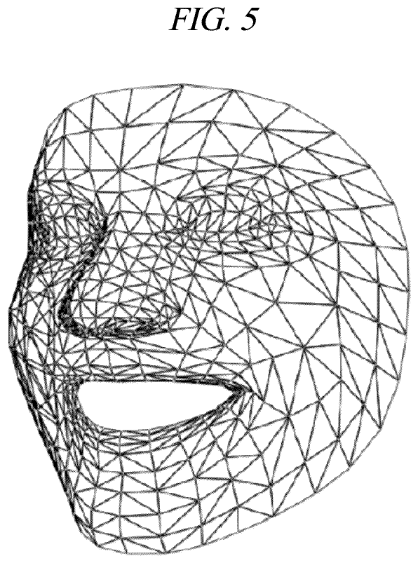

FIG. 4 and FIG. 5 are diagrams illustrating the face orientation recognition model configured to recognize the user's face orientation from a face image generated based on the determined face location.

First, based on an image 401 of the user, a face detection model 402 may extract a face image 403 of the user 20. Then, a landmark extraction model 404 may extract facial landmarks 405 from the face image 403. A convolutional neural network (CNN) may be used in this process. The face orientation recognition model may output the orientation of the face based on the positions of the eyes and nose as well as the facial contours.

Referring to FIG. 4, the face detection model 402 generates a bounding box for the face image 403 from the input image 401, and the face image 403 is input into the landmark extraction model 404 to extract the facial landmarks 405.

Referring to FIG. 5, the orientation of the face is output based on the extracted facial landmarks.

To this end, images of the user 20's face, walking scene, body shape, and clothing, or features derived from such images may be matched with the user 20 and stored in a database in which the interactive care robot 100 is installed, or in another database accessible by the interactive care robot 100 through a wired or wireless connection.

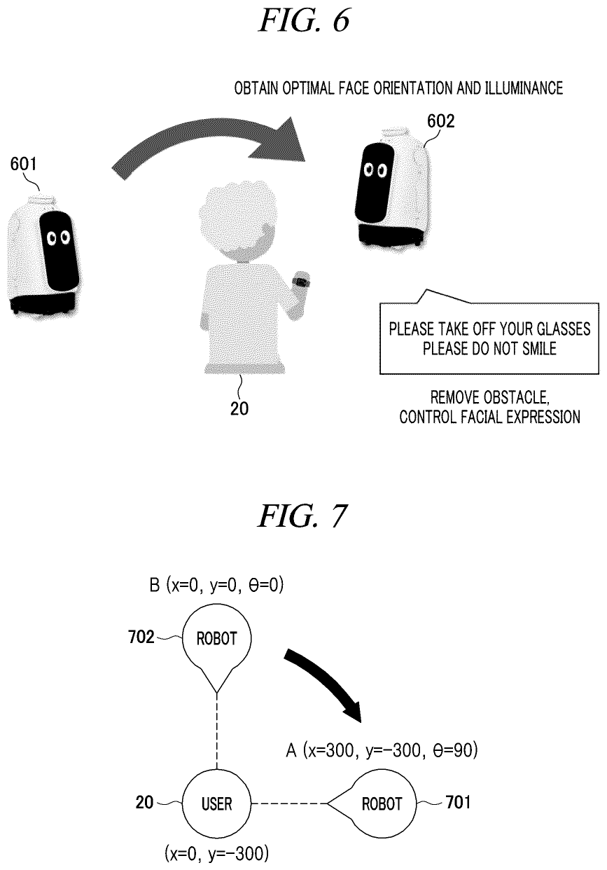

FIG. 6 illustrates an example in which the interactive care robot captures an image of the user 20 from a first position 601 and then moves to a second position 602 to capture another image of the user 20 in order to perform a more accurate situation analysis based on the captured images. The interactive care robot 100 may provide messages, such as “Please take off your glasses” or “Please do not smile” to the user 20 through voice or text output. This allows the robot to induce certain behaviors from the user 20 and to more accurately derive robot-collected information.

In order for the interactive care robot 100 to more accurately recognize the user's condition or surrounding situation and capture images, the operation controller 217 may be used to change the location or capture angle of the camera.

As described above, the operation controller 217 may include at least one of the camera controller 218 configured to control at least one of vertical rotation, horizontal rotation, and height adjustment of the camera, and the robot mover 219 configured to move the interactive care robot 100 to change its capture location.

The camera controller 218 may include a tilting unit (not shown) for rotating the camera in a vertical direction, a panning unit (not shown) for rotating the camera in a horizontal direction, and a lifting unit (not shown) for adjusting the height of the camera. The camera controller 218 may change the capture direction of the camera. The tilting unit, panning unit, and lifting unit may be directly connected to the camera, but they may also be arranged to be physically spaced apart from the camera depending on the shape or size of the interactive care robot 100. At least one of the functions of the tilting unit, panning unit, and lifting unit may be replaced by another device configured to rotate or elevate the interactive care robot 100.

After capturing an image of the user, if a recognition rate of the user's pose is equal to or less than a predetermined threshold, the interactive care robot 100 may improve the pose recognition rate by controlling the camera controller 218, which may change the capture direction of the camera by controlling at least one of the tilting unit, the panning unit, and the lifting unit.

The robot mover 219 may include components configured to move the interactive care robot 100, and may thus change the location or rotate the interactive care robot 100. Accordingly, the camera may capture an image of the user 20 from the changed location of the interactive care robot 100 to improve the pose recognition rate in the process of generating hand gesture information or full-body pose information. Further, the robot mover 219 may move the interactive care robot 100 to follow the user 20 as needed.

The robot mover 219 may provide a means for enabling the interactive care robot 100 to move within a specific space according to movement commands from a control device. More specifically, the robot mover 219 may include a motor and a plurality of wheels, which are combined to perform functions of driving, steering, and rotating the care robot 100.

FIG. 7 illustrates an example of representing, on a virtual map, the relative positions of the user 20 and the interactive care robot 100 shown in FIG. 6. The interactive care robot may calculate coordinate information between a destination B and a starting point A on the virtual map, and an angle (O) needed to face the user 20 after arriving at the destination. Thereafter, the care robot may move to coordinate information 702 of the destination. After the care robot arrives at the destination, it may rotate by the angle (O) to capture an image of the user 20.

In the example shown in FIG. 7, an interactive care robot 701 located at a position A may have coordinate values (0,0) corresponding to the position A on the virtual map, and may move to a position B after receiving coordinate values (−300, 300) of the position B. Thereafter, the care robot 701 may rotate by an input rotation angle (Θ) to capture an image of the user 20. During this process, coordinate values (e.g., −300, 0) of the user 20 and coordinate values of the care robot 701 may be continuously updated by a position tracker.

In another embodiment of the present disclosure, unlike the above-described case, the care robot may generate a virtual map solely based on imaging information generated by a vision sensor without relying on LiDAR, and may control movements of an exercise support service-providing robot. To this end, Visual SLAM (Simultaneous Localization and Mapping) technology may be used.

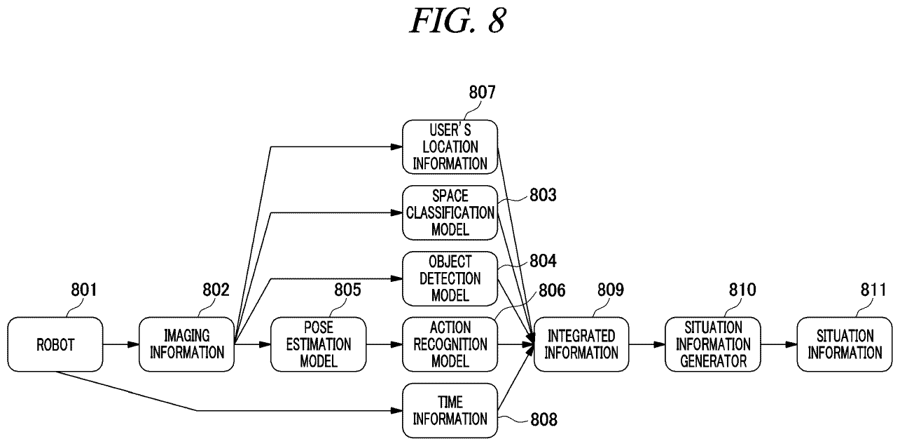

FIG. 8 is a diagram illustrating a process in which the interactive care robot 100 recognizes surrounding situations through the situation information generator 215 according to an embodiment of the present disclosure.

Referring to FIG. 8, an interactive care robot 801 captures an image of an environment around a security robot and generates imaging information 802. Then, the imaging information 802 may be input into a space classification model 803, an object detection model 804, a pose estimation model 805, and an action recognition model 806, and may be processed into element information that includes the result values from each analysis model.

Then, integrated information 809 is generated by combining the element information, the user's location information 807, and time information 808. Thereafter, the integrated information 809 is input into a situation information generator 810, which generates situation information 811 regarding the current situation.

The space classification model 803, the object detection model 804, the pose estimation model 805, and the action recognition model 806 for generating the integrated information 809 may receive the imaging information 802 generated by the camera of the interactive care robot 801 and provide predetermined outputs. These models may correspond to machine learning or deep learning models trained for graphical processing.

More specifically, the space classification model 803 may be a model configured to determine what kind of space the security robot is in based on the imaging information, and may output a class of the space being captured. A known CNN-based classification model may be used as the space classification model. To this end, CNN architectures, such as AlexNet, VGG-16, Inception, ResNet, and MobileNet, may be used.

The object detection model 804 may output a class of an object being captured based on the imaging information. The object detection model may use a CNN-based classification model to output a bounding box indicating the type and location of the detected object. To this end, CNN architectures, such as AlexNet, VGG-16, Inception, ResNet, and MobileNet, may be used. Representative object detection models may include RCNN, Fast RCNN, YOLO, Single Shot Detector (SSD), Retina-Net, and Pyramid Net.

The pose estimation model 805 may be a model configured to estimate a pose of the user or target object in an image included in the imaging information. For pose estimation, a CNN-based pose estimation algorithm may be used for 2D or 3D images to estimate 2D/3D pose information. As the pose estimation model, a CNN-based feature point detection model may be applied, and algorithms, such as MoveNet, PoseNet, OpenPose, MediaPipe, AlphaPose, Nuitrack, and Kinecct SDK, may be used. Pose information and the values calculated from it do not change over a short period of time and can be effectively used to identify a tracking target.

The action recognition model 806 may be a model configured to analyze the type of the user's action based on joint data generated by the pose estimation model 805. The action recognition model may recognize actions by using the 2D/3D pose information extracted from 2D/3D images by the pose estimation model. In this case, the action recognition model may use a rule-based method, such as a threshold-based method, to classify actions. Alternatively, the action recognition model may use a machine learning-based method, such as a Recurrent Neural Network (RNN), which is a powerful neural network for processing time-series data and suitable for action recognition based on time-series pose information, or a Graph Convolutional Network (GCN), which uses the graph structure of pose data.

That is, the situation information generator 810 may generate the situation information 811 about the surroundings of the security robot by integrating a space class, an object class, a user action, the time information 808 about the current time, and the location information 807 about the user's estimated location, all of which are derived from various models based on the imaging information 802.

Herein, the location information 807 may represent information about the location of the security robot on a smart map generated by the interactive care robot. Herein, the location information may refer to two-dimensional coordinate values relative to a specific point on an indoor map.

In addition to the method illustrated in FIG. 8, the situation information generator may also use a Large Language Model (LLM) to analyze situations around the security robot. The situation information generator 215 may input the imaging information generated by the camera of the interactive care robot 100 into the LLM, and may also convert information derived from the sensor 210 into text form to input together. That is, the situation information generator 215 may generate a prompt requesting an analysis of the user's situation based on the imaging information and sensing information, and may input the generated prompt into the LLM to receive results.

This allows a situation analysis to be performed by an external language model without allocating computing resources or capacity to the interactive care robot 100, thereby improving the processing efficiency of the interactive care robot 100. Further, as the performance of the LLM is improved, the user's situation can be analyzed more accurately.

Meanwhile, the interactive care robot 100 may generate IoT-collected information based on information collected from IoT devices connected to the interactive care robot 100.

More specifically, the interactive care robot 100 may receive various types of information from IoT devices connected thereto via wired or wireless communication. The interactive care robot 100 may be located near the IoT devices and connected thereto wirelessly via Bluetooth or infrared communication, or directly by wire. The interactive care robot 100 may also receive information, via a network, from external IoT devices that exchange information with the server connected to the interactive care robot 100.

Such IoT devices may include various smart electronics installed indoors (e.g., TVs, air conditioners, lighting devices, AI speakers, smartwatches, etc.), and each loT device may provide the interactive care robot 100 with various types of sensing information related to the user's activities or surrounding conditions (e.g., temperature, illuminance, weather, content being watched, or the user's usage history of IoT devices).

The situation information generator 215 is capable of operating while the care robot provides care services and drives within an indoor space. As the care robot drives through the indoor space, new sensing information may be generated by the sensor 210. The interactive care robot 100 may generate new situation information based on the updated sensing information to update the situation information.

The interactive care robot 100 may include the health management unit 216 configured to generate prescription information based on additional information input by the user and the user information.

Herein, the situation information may be generated by the situation information generator 215, and the additional information may be generated by the information requester 209.

The interactive care robot 100 may, through the information requester 209, ask the user about pain location and severity, abnormal symptoms and severity, meal menus, medication status, sleep information, bowel movement information, and exercise information. Based on the user's responses, the interactive care robot 100 may generate additional information, analyze the additional information, and generate prescription information corresponding to the user's current health condition through the health management unit 216.

The process by which the interactive care robot 100 asks the user about health-related matters through the information requester 209 and generates additional information based on the user's responses may be as follows. The user's responses may be input by voice or by touch on a display installed on the interactive care robot 100.

The interactive care robot 100 may ask the user about pain location and severity or abnormal symptoms and severity at a specific time each day, and may generate related additional information based on the user's responses.

The interactive care robot 100 may ask the user about sleep information (such as whether they woke up during the night, whether they slept well, or whether they feel tired in the morning) every morning, and may generate additional information based on the user's responses.

When the interactive care robot 100 determines from the generated situation information that the user is eating or has just finished a meal, it may ask the user about the meal menu and alcohol consumption, and may generate related additional information based on the user's responses.

The interactive care robot 100 may ask the user about their medication status when a scheduled medication time has passed, and may generate related additional information based on the user's responses.

The interactive care robot 100 may ask the user about their bowel movement condition when it determines from the situation information that the user has come out of the bathroom, and may generate related additional information based on the user's responses.

The interactive care robot 100 may ask the user about their daily exercise information (type, duration, etc.), and may generate related additional information based on the user's responses.

The interactive care robot 100 may generate additional information when health-related user information is recognized during a conversation between the user and the robot.

Further, the interactive care robot 100 may obtain the user's meal menu, medication status, and exercise information through the camera without using voice recognition.

The process by which the interactive care robot 100 generates prescription information based on additional information input by the user and existing user information may be as follows.

Herein, the user information used to generate prescription information may include data, such as the user's gender, age, and medical conditions, and it may be assumed that the interactive care robot 100 has already obtained related information.

If the user mentions a pain area, the interactive care robot 100 may ask about the severity of pain. If it is deemed unbearable, the robot may notify a guardian of an emergency and perform an emergency call service. However, if the pain is deemed tolerable, the robot may generate and provide prescription information recommending foods or exercises beneficial for the pain area. The user may express the severity of pain on a scale from 0 to 10.

If the user mentions a symptom area, the interactive care robot 100 may ask about the severity of symptom. If it is deemed unbearable, the robot may notify the guardian of an emergency and perform an emergency call service. If the symptom is deemed tolerable, the robot may generate and provide prescription information recommending foods or exercises beneficial for the symptom area. The user may express the severity of symptom on a scale from 0 to 10.

If the user mentions a meal menu, the interactive care robot 100 may determine whether the meal is appropriate considering the user's medical conditions, and may inform the user. The interactive care robot 100 may also generate prescription information to recommend menus suitable for the user's medical conditions for the next meal.

If the user mentions medication status and has not taken the medication, the interactive care robot 100 may generate prescription information to promote medication or inform the user about its importance.

If the user mentions sleep information and the quality of sleep is poor, the interactive care robot 100 may generate and provide prescription information recommending foods or exercises that help improve sleep.

If the user mentions bowel movement information and their bowel movement condition is poor, the interactive care robot 100 may generate prescription information recommending a hospital visit depending on the severity or recommending foods or exercises beneficial for bowel movements.

The interactive care robot 100 may digitize the stored user information including basic information, pain areas, abnormal symptoms, meal menus, medication status, sleep information, bowel movement information, and exercise information, and may analyze the relationships among them to generate and provide health information and recommendations as prescription information. For example, the interactive care robot 100 may link the meal menu with basic information, pain, abnormality, and bowel movement information to generate and provide prescription information indicating the cause of the problem and recommending menus. Further, the interactive care robot 100 may link pain areas and abnormal symptoms with exercise information to generate and provide prescription information indicating that the problem may have been caused by exercise. As another example, the interactive care robot 100 may link medication status with pain areas and abnormal symptoms to generate and provide prescription information indicating that the problem may have been caused by not taking the medication. Furthermore, the interactive care robot 100 may link sleep information with abnormal symptoms to generate and provide prescription information indicating that the problem may have been caused by sleep.

The interactive care robot 100 may predict the user's health condition by identifying changing patterns over time in the stored user information including basic information, pain areas, abnormal symptoms, meal menus, medication status, sleep information, bowel movement information, and exercise information. For example, if the user has abdominal pain and the bowel movement cycle is getting longer, the interactive care robot 100 may generate prescription information recommending a visit to an internal medicine clinic. If pain or abnormal symptoms worsen without improvement, the interactive care robot 100 may generate prescription information recommending a relevant hospital. If the user continues not to exercise, the interactive care robot 100 may generate prescription information to promote exercise.

The interactive care robot 100 may also input the stored user information including basic information, pain areas, abnormal symptoms, meal menus, medication status, sleep information, bowel movement information, and exercise information into an AI model to predict the user's future health. Herein, the AI model may include machine learning/deep learning-based models and large language models. The output of the AI model may include a health score, a health risk level, a hospital visit history, recommended hospitals, recommended exercises, recommended menus, and recommended activities.

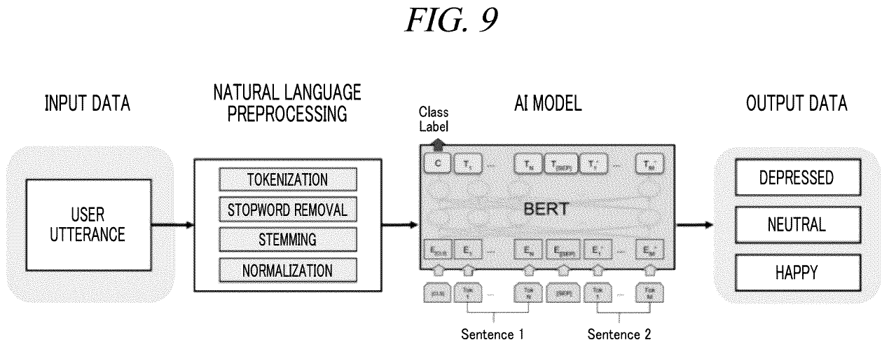

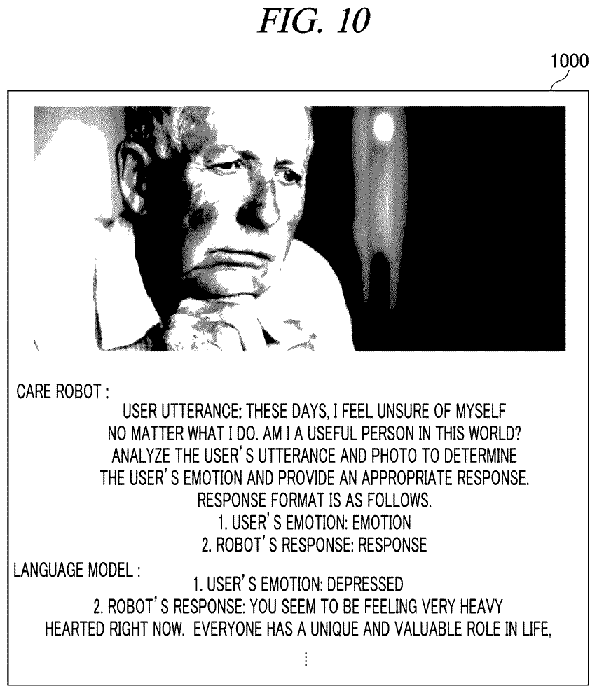

FIG. 9 and FIG. 10 are diagrams illustrating a process of analyzing the user's emotion by the emotion information generator according to an embodiment of the present disclosure.

The interactive care robot 100 may further include the emotion information generator 220 configured to derive emotion information of the user from conversation history information and utterance information generated by a microphone included in the sensor 210.

The emotion information generator 220 may input the conversation history information and additional information into the language model to derive the emotion information. Herein, the additional information may be generated by the information requester 209.

The emotion information generator 220 may also input the conversation history information, the additional information, and user images captured by the camera of the sensor 210 into the language model to derive the emotion information.

The interactive care robot 100 may analyze the user's emotions during conversations and provide responses or care services corresponding to the emotions, thereby enabling emotional care of the user.

The emotion information generator 220 may generate emotion information of the user by using at least one of the conversation history information, the user's voice, and the user's facial expressions.

The emotion information generator 220 may classify the user's emotions from the conversation history information based on the user's utterance sentences. The emotion information generator 220 may use the information requester 209 to directly ask the user about their emotions or may indirectly infer them.

When the emotion information generator 220 directly asks the user about their emotions through the information requester 209, it may, for example, ask questions, such as “How are you feeling today?” or “Did anything fun happen today?” at certain times or in certain situations each day, and may generate emotion information based on the user's responses. In this case, although the interactive care robot 100 can obtain clear answers from the user, the user may feel reluctant to talk to the robot. Therefore, the interactive care robot 100 may generate utterances that can more indirectly infer the user's emotions from the conversation history information. For example, if the user says, “I went to the senior center today,” the interactive care robot may respond,

“You went to the senior center. I bet it felt nice to visit again after such a long time.” Alternatively, the interactive care robot may generate appropriate utterances using the user's situation information or daily pattern information derived by the situation information generator 215. The inquiry sentences of the interactive care robot 100 and the user's utterance sentences may be input into the AI model together, and the emotion information generator may generate the emotion information based on an output from the AI model.

The emotion information generator 220 may input the entire conversation history information into the language model to generate emotion information that classifies momentary emotions. In this process, generative AI such as an LLM may be used to understand the contextual meaning of the conversation.

The emotion information generator 220 may input the user's utterance sentences into the AI model after preprocessing, such as tokenization, stopword removal, stemming, and normalization. Examples of the AI model may include BERT (Bidirectional Encoder Representations from Transformers), GPT (Generative Pre-trained Transformer), XLNet, LSTM (Long Short-Term Memory), GRU (Gated Recurrent Unit), and RNN (Recurrent Neural Network).

The emotion information generator 220 may use an AI model to generate emotion information based on the user's voice tone from the user's utterance information input through the microphone. Examples of the AI model may include CNN (Convolutional Neural Network), LSTM, GRU, RNN, Deep Belief Network, and Transformer.

FIG. 9 is a diagram illustrating a process of generating emotion information, including a process 901 of using the user's utterance as input data, a preprocessing process 902 for the user's utterance, a process 903 of inputting the preprocessed user utterance into the AI model, and a process 904 of generating emotion information based on an output from the AI model.

The emotion information generator 220 may more accurately identify the user's emotions based on user images captured by the camera of the sensor 210. The emotion information generator 220 may input the user images obtained through the camera into an AI-based facial expression recognition model to derive facial expression states.

The emotion information generator 220 may also classify emotions by inputting two or more of the conversation history information, utterance information, and user images into the AI model. In particular, text, image, and voice information can be input into an LLM simultaneously, and, thus, the emotion information generator 220 may simultaneously analyze the user's utterance, voice, and facial expressions to generate emotion information.

FIG. 10 is a diagram illustrating an example in which conversation history information and user images are input into an LLM, and answers are generated by the LLM.

The emotion information generated by the emotion information generator 220 may be uploaded to the server and reflected in the conversations of the interactive care robot 100 in real time. For example, if the emotion information classifies the user's emotion as “depressed,” the interactive care robot 100 may switch to a mode for responding to “depressed” emotions and appropriately select related responses and care services to provide.

The interactive care robot 100 may display different facial expressions according to the generated emotion information. For example, if the interactive care robot 100 is equipped with a display or device functioning as a face, it may show a happy or comforting expression to the user by controlling the display or device.

When the generated emotion information indicates that the user's emotion is negative, the interactive care robot 100 may conduct counseling with the user using an LLM. In this case, the interactive care robot 100 may fine-tune the LLM in advance to optimize it for emotional care and counseling. The interactive care robot 100 may also prepare several fine-tuned language models, each tailored to a specific user emotion, and may switch between the models depending on the detected user emotion.

The user's emotions may be continuously uploaded to the server by the interactive care robot 100, and the robot may provide appropriate services by analyzing emotional changes over a certain period. For example, if the interactive care robot 100 detects that the user's emotions are frequently negative, such as depression or anxiety, over a week, it may provide care services, such as notifying the guardian or connecting the user with a professional counselor.

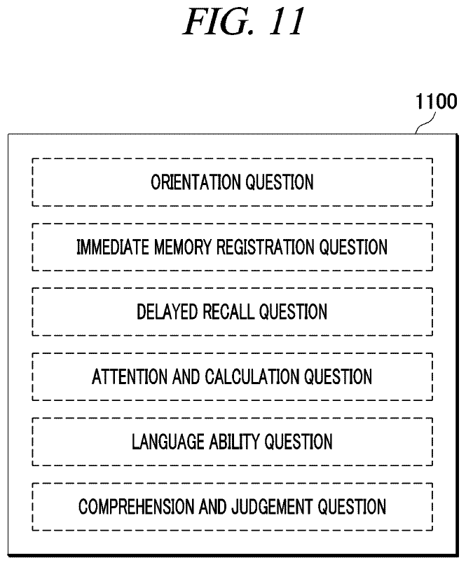

FIG. 11 is a diagram illustrating a process of analyzing the user's cognitive ability by a cognitive information generator according to an embodiment of the present disclosure.

More specifically, FIG. 11 illustrates examples of questions used by the interactive care robot 100 to evaluate the user's cognitive ability.

The interactive care robot 100 may include the cognitive information generator 221 configured to conduct a dementia test or evaluate cognitive ability through conversations with the user, and to generate cognitive information by digitizing and analyzing the collected information to assess dementia risk.

The interactive care robot 100 may provide the user with a cognitive impairment screening questionnaire, such as the Korean Dementia Screening Questionnaire (KDSQ), Samsung Dementia Questionnaire (SDQ), Attention Questionnaire (AQ), and Seoul Informant Report Questionnaire for Dementia (SIRQD), and may store the results on the server and provide alerts to the user and guardian when a risk is detected.

Meanwhile, the interactive care robot 100 may conduct a brief cognitive ability test through conversations with the user. For example, as a basic test, the interactive care robot 100 may ask the user about the current day of the week, date, season, and location, and may verify the answers by voice recognition or screen touch to determine dementia risk and generate cognitive information.

The interactive care robot 100 may also provide the user with quizzes related to cognitive functions as a quiz test, and may generate cognitive information based on the user's answers. Since the interactive care robot 100 has already stored basic user information, it may also create quizzes based on that information.

The interactive care robot 100 may evaluate cognitive functions by asking the user to repeat difficult-to-pronounce words and recognizing them by voice recognition, or by telling the user something to remember and asking about it after a certain time has passed, and may generate cognitive information based on the evaluation.

Furthermore, the interactive care robot 100 may generate cognitive information by determining, through daily conversations with the user, whether the user repeatedly says the same words or makes logically inconsistent statements. The interactive care robot 100 may digitize the results of cognitive function tests and cognitive ability scores and upload them to the server. Such data may be provided to the user and the guardian.

The cognitive information generated by the cognitive information generator 221 may be configured to provide alerts when the user has a cognitive impairment or when persistent problems are detected during conversations with the user.

The above-described method of providing an interactive care service performed by the interactive care robot can be implemented as a computer program stored in a computer-readable storage medium to be executed by a computer or a storage medium including instructions executable by a computer. Also, the above-described method can be implemented as a computer program stored in a computer-readable storage medium to be executed by a computer.

A computer-readable recording medium can be any usable medium which can be accessed by the computer and includes all volatile/non-volatile and removable/non-removable media. Further, the computer-readable recording medium may include all computer storage media. The computer storage media include all volatile/non-volatile and removable/non-removable media embodied by a certain method or technology for storing information such as computer-readable instruction code, a data structure, a program module or other data.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

### EXPLANATION OF CODES

100: Care robot
201: Conversation history information generator
202: Information processor
206: Priority setting unit
209: Information requester
210: Sensor
211: Image analyzer
212: Storage period setting unit
215: Situation information generator
216: Health management unit
217: Operation controller
220: Emotion information generator
221: Cognitive information generator

We claim:

1. An interactive care robot, comprising:
at least one sensor including a microphone configured to receive voice conversations with a user;
a memory configured to store instructions; and
a processor operatively connected to the memory and configured to execute the instructions,
wherein the processor:
generates conversation history information based on the conversations with the user received through the microphone;
generates user information from the conversation history information and classifies the user information into predetermined categories;
sets a priority of a care service to be provided to the user based on the classified user information and the conversation history information; and
selects a care service to be provided to the user based on the set priority and controls components of the interactive care robot to perform the selected care service.

2. The interactive care robot of claim 1,
wherein the processor generates the conversation history information by converting the voice conversations between the user and the interactive care robot into text.

3. The interactive care robot of claim 1,
wherein in the process of generating user information,
the processor:
extracts utterance sentences of the user from the conversation history information and preprocess the extracted sentences;
derives entities from the preprocessed utterance sentences through an entity recognition algorithm and assigns attributes to the entities; and
maps the entities within the utterance sentences based on the attributes of the entities and generates the user information.

4. The interactive care robot of claim 1,
wherein in the process of setting a priority,
the processor uses a rule-based algorithm to set a priority of the user information based on predetermined priorities for the respective categories.

5. The interactive care robot of claim 1,
wherein in the process of setting a priority,
the processor:
derives daily pattern information and preference information of the user from the conversation history information; and
uses an AI-based priority setting model that receives the user information, the daily pattern information, and the preference information to output a priority for the user information.

6. The interactive care robot of claim 5,
wherein in the process of setting a priority,
the processor sets priorities by using the AI-based priority setting model while also setting predetermined priorities for user information classified into predetermined categories.

7. The interactive care robot of claim 1,
wherein the processor is further configured to request additional information from the user based on the classified user information.

8. The interactive care robot of claim 7,
wherein in the process of requesting additional information,
the processor selects at least part of the user information based on the priority, and requests the additional information based on the selected user information.

9. The interactive care robot of claim 7, further comprising:
a camera,
wherein the processor detects an object in an image captured by the camera and detects a place of capture, and determines, based on features of the detected object and the place of capture, whether the detected object is a newly added object, and
when the detected object is determined to be a newly added object, the processor is further configured to request additional information about the detected object from the user.

10. The interactive care robot of claim 1,
wherein the processor is further configured to set, in the user information, a storage period indicating a duration to be reflected in the conversations with the use.

11. The interactive care robot of claim 10,
wherein in the process of setting a storage period,
the processor uses a rule-based algorithm to set a storage period of the user information based on predetermined storage periods for the respective categories.

12. The interactive care robot of claim 10,
wherein in the process of setting a storage period,
the processor:
derives daily pattern information and preference information of the user from the conversation history information; and
uses an AI-based storage period setting model that receives the user information, the daily pattern information, and the preference information to output a storage period for the user information.

13. The interactive care robot of claim 1,
wherein the processor:
generates situation information about surrounding conditions of the user based on sensing information obtained from the sensor;
requests additional information from the user based on the situation information; and
generates prescription information based on additional information input by the user and the user information.

14. The interactive care robot of claim 13,
wherein to generate the situation information,
the processor:
controls at least one of vertical rotation, horizontal rotation, and height adjustment of the camera; or
controls movements of the interactive care robot to change a capture location of the interactive care robot.

15. The interactive care robot of claim 1,
wherein the processor is further configured to derive emotion information of the user from the conversation history information and the user's utterance information received through the microphone.

16. The interactive care robot of claim 15,
wherein the processor:
requests additional information from the user based on the classified user information; and
inputs the conversation history information and the additional information into a language model to derive the emotion information.

17. The interactive care robot of claim 16, further comprising:
a camera,
wherein in the process of deriving the emotion information,
the processor inputs the conversation history information, the additional information, and user images captured by the camera into the language model to derive the emotion information.
